(19) United States
(12) Patent Application Publication
(10) Pub. No.: US 2003/0125639 A1
(43) Pub. Date: Jul. 3, 2003

(54) Biopsy needle having rotating core for shearing tissue

(76) Inventors: John S. Fisher, Belleair, FL (US); Frederick Ahari, Tucson, AZ (US)

Correspondence Address: SMITH & HOPEN PA, 15950 BAY VISTA DRIVE, SUITE 220, CLEARWATER, FL, 33760

(21) Appl. No.: 09/683,454

(22) Filed: Jan. 2, 2002

(51) Int. Cl. A61B 10/00

(52) U.S. Cl. 600/564

(57) ABSTRACT

A biopsy tool that performs a biopsy and a complete removal of a tumor or lesion in a single step. A hollow pointed needle having a cylindrical lumen has an inner tube mounted for rotation in the lumen. A slot is formed in the hollow needle in a sidewall of the needle and an opening having sharp peripheral edges is formed in the inner tube. A vacuum is applied to the lumen of the hollow needle to pull tissue into the slot during a biopsy procedure. Rotation of the inner tube shears off a slice of tissue and the vacuum pulls the slice of tissue to a remote collection point. Each rotation of the inner tube removes an additional slice of tissue so that an entire lesion or tumor is removed in a short period of time. The sliced tissue is undamaged and suitable for use as a biopsy sample for diagnostic purposes.

### BACKGROUND OF INVENTION

1. Field of the Invention

This invention relates, generally, to biopsy needles. More particularly, it relates to a biopsy needle that cuts and collects large quantities of tissue in a short period of time.

2. Description of the Prior Art

Biopsy needles are used to cut small samples from lesions or tumors in soft tissue so that the samples may be analyzed in a laboratory for diagnostic purposes. If the lab results indicate that a lesion or tumor should be removed, a surgical procedure is required. Thus, the patient must undergo two procedures.

If the first sample taken is insufficient in size, then the patient must undergo a second biopsy. Thus, the patient must undergo three procedures if the first biopsy returns an insufficient amount of tissue and the lab results from a second biopsy indicate that surgical removal is required.

What is needed, then, is an improved biopsy tool that enables a physician to always collect a sufficient amount of tissue during a first biopsy so that a repeat biopsy need not be performed.

If a biopsy tool having the ability to remove very large quantities of tissue during a biopsy procedure could be created, then the step of performing a post-biopsy surgical removal of the lesion or tissue could be eliminated and the patient would have but one procedure to undergo.

Tools are known that can remove large quantities of tissue in a short amount of time, but the tools are not suitable for use in performing biopsies because they grind up and tear the removed tissue, rendering it unsuitable for use as a biopsy sample. They are also too large to be used in lungs and some other organs. Moreover, they are blunt at their distal free end because they burrow into tissue in much the same way as a tunnel-digging machine burrows into a mountain. Accordingly, they cannot be inserted deep into soft tissue and used to collect biopsy samples.

In view of the prior art considered as a whole at the time the present invention was made, it was not obvious to those of ordinary skill in the art that providing a biopsy needle capable of removing entire lesions or tumors in one procedure was desirable because conventional wisdom has always held that biopsy needles should remove only enough tissue to enable laboratory analysis thereof.

Therefore, it was not obvious how a biopsy tool capable of removing large quantities of material could be provided. Nor was it obvious how undamaged samples could be obtained in large quantities.

### SUMMARY OF INVENTION

The long-standing but heretofore unfulfilled need for a biopsy needle having the capability of removing large, undamaged quantities of lesions or tumors during a biopsy procedure is now met by a new, useful, and nonobvious invention. The novel biopsy tool of this invention includes a hollow needle having a cylindrical lumen and a pointed distal end. A slot of elongate, longitudinally-extending configuration is formed in a cylindrical side wall of the needle. The slot has a distal end disposed proximal to the pointed distal end of the needle.

An inner tube of hollow cylindrical configuration is disposed within the lumen of the hollow needle. A rotation means is provided for rotating the inner tube about its longitudinal axis.

An opening having sharp peripheral edges is formed in the inner tube; the opening is in intermittent registration with the slot as the inner tube rotates about its longitudinal axis.

A vacuum source is in fluid communication with the lumen of the hollow needle so that tissue is pulled into the slot by the vacuum. Tissue pulled into the slot by the vacuum is sheared off by a sharp peripheral edge of the opening formed in the inner tube as the opening rotates past the slot.

A quantity of tissue is cleanly sheared or sliced off by the sharp peripheral edge during each rotation of the inner tube. Each piece of sliced off tissue is pulled toward the vacuum source so that the slot and opening are cleared of tissue for each rotation of the inner tube. The inner tube rotates at a high rotational speed (angular velocity) so that the biopsy tool removes a large quantity of tissue in a brief amount of time.

Accordingly, the biopsy tool eliminates a need to perform a biopsy and a tissue removal procedure in two separate steps. The speed of rotation is under the control of the user-physician because some applications will require differing speeds. For example, if a morcellation function is required, such as cutting prostate tissue to treat BHP disease, the rotation is sped up as much as possible to enable cutting as much tissue as possible in a short period for time. However, where samples of relatively large size are required, the speed of rotation is reduced to enable the collection of intact tissue samples.

The novel structural design of this invention enables small needles (up to 18 gauge) to be employed in biopsy procedures where large biopsy needles are not recommended, such as in the biopsy of a lung nodule. A small biopsy needle performs tissue resection by actually cutting the tissue in such applications.

An important object of this invention is to provide a biopsy tool capable of removing large quantities of tissue during a biopsy procedure.

A closely related object is to provide a biopsy needle capable of removing an entire or nearly an entire lesion or tumor during a biopsy procedure.

Another important object is to provide a biopsy needle that provides cleanly cut samples of the lesion or tumor to facilitate the work of a laboratory.

These and other important objects, advantages, and features of the invention will become clear as this description proceeds.

The invention accordingly comprises the features of construction, combination of elements, and arrangement of parts that will be exemplified in the description set forth hereinafter and the scope of the invention will be indicated in the claims.

### BRIEF DESCRIPTION OF DRAWINGS

For a fuller understanding of the nature and objects of the invention, reference should be made to the following detailed description, taken in connection with the accompanying drawings, in which:

FIG. 1 is a diagrammatic view depicting all of the parts of a first embodiment of the invention in their operable interconnection;

FIG. 2 is a side elevational view of a hollow needle that forms a part of the novel biopsy tool of this invention;

FIG. 3 is a side elevational view of an inner tube that is mounted for rotation within the lumen of the hollow needle of FIG. 1;

FIG. 4 is a side elevational view depicting the inner tube when positioned within the lumen of the hollow needle;

FIG. 5 is a diagrammatic view depicting a small liquid flow tube that forms a part of the hollow needle;

FIG. 6 depicts an alternative shape of an opening formed in the inner tube;

FIG. 7A is a top view of a second embodiment of the invention when the balloon of the second embodiment is deflated;

FIG. 7B is a bottom view of the second embodiment when said balloon is deflated;

FIG. 7C is a bottom view of the second embodiment when the balloon is inflated;

FIG. 7D is an end elevational view of the second embodiment when the balloon is inflated;

FIG. 7E is a longitudinal sectional view of the second embodiment with the balloon inflated;

FIG. 8A is a top plan view of a third embodiment of the invention, depicting a cutting tube in housing relation to the hollow needle;

FIG. 8B is a longitudinal sectional view of said third embodiment and is the first view in a three view animation depicting the operation of the third embodiment;

FIG. 8C is the second view in said three view animation;

FIG. 8D is the third view in said three view animation;

FIG. 9A is a longitudinal sectional view of a fourth embodiment and is the first view of a three view animation depicting the operation of the fourth embodiment;

FIG. 9B is the second view in said three view animation;

FIG. 9C is the third view in said three view animation; and

FIG. 10 is a longitudinal sectional view of a fifth embodiment.

### DETAILED DESCRIPTION

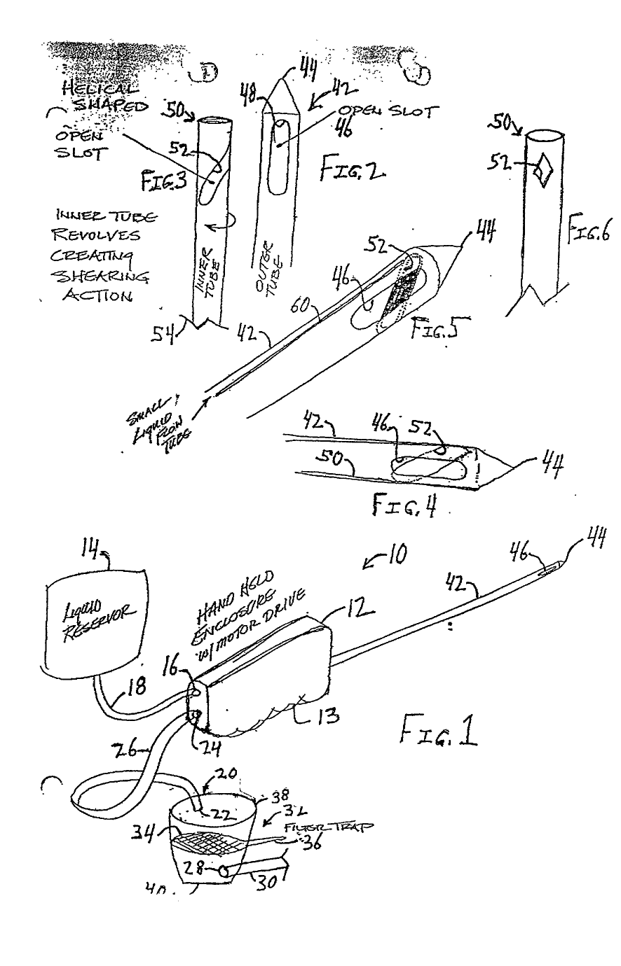

Referring to FIG. 1, it will there be seen that the reference numeral 10 denotes an illustrative embodiment of the present invention as a whole.

Assembly 10 includes a portable, hand-held housing 12 for a direct current motor, not shown. Finger grips 13 are provided on a lower edge of housing 12 to facilitate gripping thereof by a user's hand.

Liquid reservoir 14 is connected to inlet port 16 formed in the proximal end of housing 12 by hose 18.

Receptacle 20 has an inlet port 22 connected to outlet port 24 formed in said housing proximal end by hose 26. Outlet or vacuum port 28 of receptacle 20 is connected to a remote vacuum source, not shown, by hose 30.

Filter trap 32 has a main body 34 formed of a filter material and a handle 36 secured to said main body. Main body 34 is positioned within receptacle 20 at a point below rim 38 and above imperforate bottom wall 40 thereof.

Receptacle 20 has a horizontal slot formed therein to enable insertion of said main body 34 into its depicted position and withdrawal of said main body so that it can be transported to a laboratory.

Hollow needle 42 is depicted in FIG. 1 but its construction is best disclosed in connection with FIG. 2. Needle 42 includes a pointed distal free end 44 that facilitates insertion of the needle into tissue. An elongate slot 46 is formed in a cylindrical side wall of needle 42; distal end 48 of slot 46 is proximal to pointed end 44. Slot 46 has a longitudinal axis of symmetry that is parallel to a longitudinal axis of needle 42. The lumen of needle 42 is cylindrical in configuration.

Hollow needle 42 is positioned beside a suspected lesion or tumor when the novel tool is used, i.e., needle 42 is positioned in close laterally spaced relation to said lesion or tumor so that said lesion or tumor may enter into slot 46 when said lesion or tumor is subjected to a vacuum emanating from said slot or when said lesion or tumor is pushed into said slot by physical means of the type disclosed in the second embodiment of this invention, disclosed hereinafter.

A hollow inner tube is denoted 50 as a whole in FIG. 3. It has an external diameter slightly less than an internal diameter of needle 42 so that it fits within the lumen of needle 42. In the first embodiment of the invention, a helical opening 52 is formed in inner tube 50, near its distal free end. The peripheral edges of opening 52 are sharp.

Proximal end 54 of inner tube 50 is connected by a suitable interconnecting means, not shown, to the output shaft of the motor, not shown, housed within housing 12. Thus, as the output shaft of the motor rotates, inner tube 50 rotates conjointly therewith. Such rotation brings helical opening 52 into registration with slot 46 once per revolution.

FIG. 4 depicts inner tube 50 disposed within the lumen of hollow needle 42. Whereas slot 46 is parallel to the longitudinal axis of needle 42 as aforesaid, helical opening 52 is oblique to the longitudinal axis of inner tube 50. Thus, as opening 52 rotates past slot 46, the angular difference between opening 52 and slot 46 enhances the efficiency of the shearing action of the sharp peripheral edges of opening 52, in much the same way as a pair of scissors severs an item being cut at an angle.

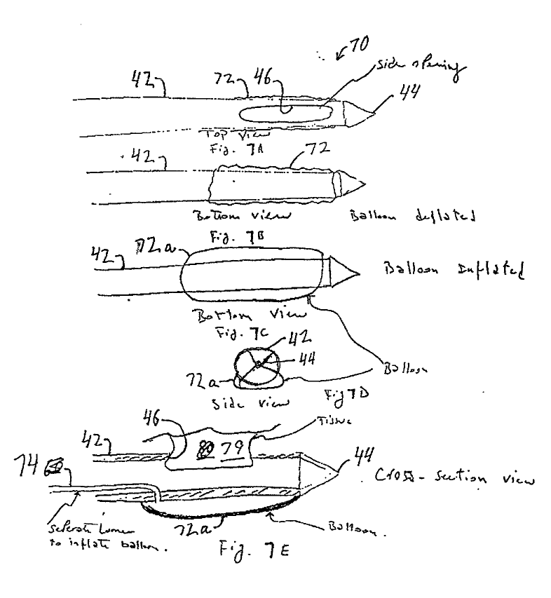

Opening 52 may be straight instead of helical as indicated in FIG. 5. As depicted in FIG. 5, the axis of opening 52 is oblique to the axis of slot 46 to provide a scissors-like shearing action.

A small liquid flow tube 60 (FIG. 5) may be mounted on an exterior surface of the cylindrical side wall of hollow needle 42, or within the lumen of said hollow needle, or it may be formed within the cylindrical side wall of said hollow needle. The proximal end of flow tube 60 is in water-tight fluid communication with port 16 (FIG. 1) formed in housing 12 and the distal free end of said flow tube is positioned near slot 48 to irrigate the site of the shearing action. Thus, water, saline solution, or other suitable irrigating fluid, is delivered to flow tube 60 by reservoir 14 through hose 18.

When the remote vacuum source, not shown, is activated, hose 30 transmits that vacuum to receptacle 20 at vacuum port 28. Since main body 34 of filter trap 32 is formed of a mesh material, the vacuum is transmitted through said main body 34 and through port 22 to hose 26 to port 24 of housing 12. Port 24 is in air-tight communication with the lumen of hollow needle 42 so the vacuum then appears in said lumen and tissue is pulled into said lumen through slot 46 by said vacuum.

When inner tube 50 is rotated within the lumen of hollow needle 42, opening 52 enters into in registration with slot 46 once per revolution as aforesaid. Accordingly, the sharp peripheral edges of opening 52 slice off the tissue pulled into slot 46 by the vacuum. As opening 52 rotates away from slot 46, additional tissue is pulled into slot 46 by the vacuum so that said additional tissue is sliced off when opening 52 again enters into registration with slot 46. Each slice of tissue is cut cleanly by the shearing action of the sharp peripheral edges so that each slice is suitable for use as a sample or specimen in a diagnostic laboratory. The vacuum pulls each piece of sheared off tissue to filter trap main body 34. The vacuum also draws the irrigation fluid and pulls it toward the source of the vacuum.

Due to the high speed of rotation of inner tube 50, a very large quantity of tissue may be sheared off and vacuumed to main body 34 of filter trap 32 in a very short period of time. Thus, the entire lesion or tumor may be entirely removed during the biopsy procedure, thereby eliminating the need for surgery in the event the laboratory analysis of the samples indicates that removal of the lesion or tumor is necessary. If surgical removal is not necessary, no harm is done in removing the lesion or tumor.

The shape of opening 52 is not limited to a helical or a straight shape. As indicated in FIG. 6, for example, opening 52 could have a diamond shape. Any geometrical shape, such as a corkscrew shape, a sinusoidal shape, a sawtooth shape, etc., for opening 52 is acceptable as long as it performs a clean shearing action as its sharp peripheral edges rotate past slot 46.

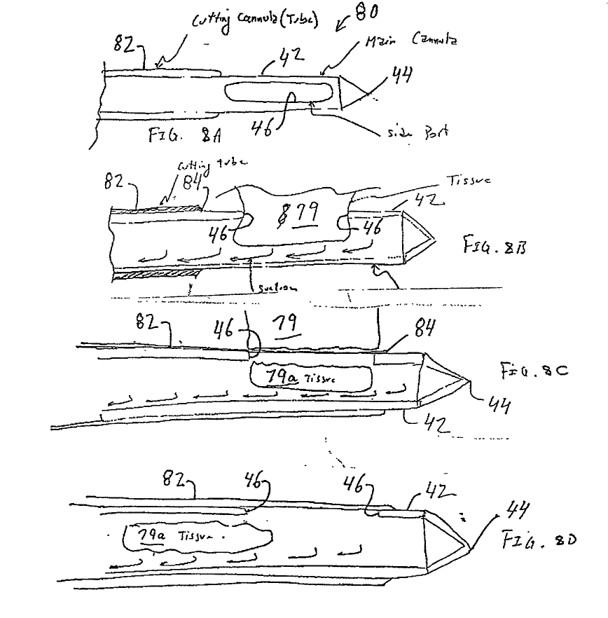

FIGS. 7A-7E depict a second embodiment of the novel biopsy tool. This second embodiment is denoted 70 as a whole. It has the same structure as the first-described embodiment, but it adds balloon 72 thereto and it may or may not include a vacuum means. Specifically, balloon 72 is positioned on an external surface of hollow needle 42 in diametric opposition to slot 46. A suitable inflation means for selectively inflating the balloon includes lumen 74 (FIG. 7E) and a remote source of compressed air or a non-compressible fluid such as a saline solution.

In FIGS. 7A and 7B, balloon 72 is depicted in its deflated condition. In FIGS. 7C-7E, the balloon is inflated and is denoted 72a. When inflated, as perhaps best understood in connection with FIG. 7E, balloon 72a presses against the patient's tissue and urges slot 46 toward lesion or tumor 79 so that said lesion or tumor 79 protrudes into slot 46 as depicted in said FIG. 7E. The sharp cutting edge 52 of inner tube 50 then severs that part of lesion or tumor 79 that protrudes into said slot.

Balloon 72, when inflated as at 72a, is thus understood to perform essentially the same function as a vacuum in that it serves to position the lesion or tumor into slot 46. Thus, it is clear that the balloon arrangement of this second embodiment may be used in lieu of the vacuum means of the first embodiment or in conjunction therewith.

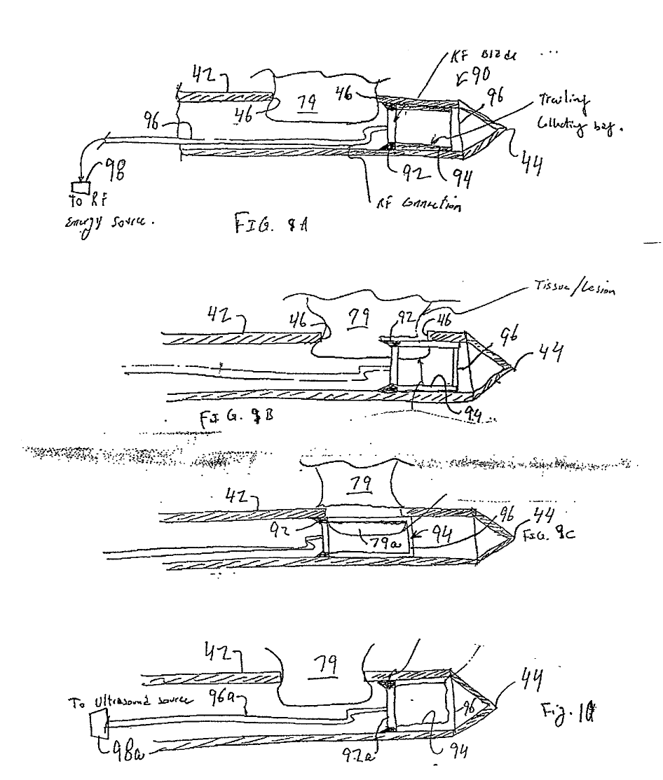

A third embodiment of the invention is depicted in FIGS. 8A 8D and is denoted 80 as a whole.

This third embodiment eliminates inner tube 50. Instead, the cutting function is performed by a cutting cannula 82 having an inner diameter or lumen sufficient to slidingly receive hollow needle 42 as depicted in FIG. 8A. As in the earlier embodiments, the biopsy or tissue removal procedure begins with the step of positioning slot 46 in closely laterally spaced relation to a lesion or tumor 79. Cutting cannula 82 is positioned in a retracted configuration as depicted in said FIG. 8A so that slot 46 is fully uncovered.

Note in FIG. 8B that the leading end 84 of cutting cannula 82 is sharp to facilitate severing of a lesion or tumor 79 that has been pulled into the lumen of hollow needle 42 though slot 46 by a vacuum.

The cutting stroke is depicted in FIG. 8C; a slice of lesion or tumor 79 has been cleanly severed and is denoted 79a. A motor means and gear assembly, not shown, is used to drive cutting cannula 82 in a proximal-to-distal direction to accomplish the severing of said lesion or tumor. A bias means could also be employed to drive cutting cannula 82 into the tumor or lesion. Moreover, cutting cannula 82 may be rotated about its longitudinal axis as it is driven into the tissue. The position of cutting cannula 82 depicted in FIG. 8C is its extended position.

FIG. 8D depicts the removal under vacuum of slice 79a to filter trap 32 (FIG. 1). Note that cutting cannula 82 remains in its extended position during the removal of slice 79a. This maintains the vacuum inside the hollow interior of outer needle 42. After slice 79a has been collected in the filter trap, the motor and gear assembly, or a suitable bias means, cause retraction of cutting cannula 82 to its FIG. 1 position and the above-described cycle is repeated until the lesion or tumor 79 has been sliced into a plurality of undamaged specimens and removed in part or entirely from the patient. The speed of the motor means is under the control of the user-physician so that the speed of reciprocation of cutting cannula 82 between its retracted and extended positions as well as its speed of rotation, if desired, is selected by the physician. In this way, as already mentioned, a biopsy procedure may become a lesion or tumor removal procedure at the option of the physician.

FIGS. 9A-9C depict a fourth embodiment of the invention, denoted 90 as a whole. Annular RF blade 92 having a sharp trailing edge for slicing tissue is initially slideably positioned within the cylindrical lumen of hollow needle 42 at a location distal of slot 46, as depicted in FIG. 9A. The FIG. 9A position is the extended position of annular blade 92. A retrievable collecting bag preferably in the form of an expandable, accordion-like container 94 for capturing a severed specimen 79a of a lesion or tumor 79 is positioned in leading relation to said sharp trailing edge and is connected thereto for conjoint movement therewith. The imperforate bottom of bag 94 is denoted 96, it being understood that the opposite end or mouth of bag 94 is open. Annular RF blade 92 having the sharp cutting edge is positioned at said mouth of RF bag 94.

An RF connection 96 has a first end in communication with a remote RF energy source 98 and a second end is connected to annular RF blade 92. Upon activation of the RF energy source, RF blade 92 and collecting bag or container 94 are slidingly displaced by the vacuum in a distal-to-proximal direction relative to stationary hollow needle 42 as understood by comparing FIG. 9B with FIG. 9A. The FIG. 9B position of RF blade 92 is its extended position. This sliding displacement enables the sharp cutting edge of RF blade 92 to sever a slice 79a of lesion or tumor 79 that is protruding through slot 46 into the cylindrical lumen of hollow needle 42. The heat generated by the radio frequency cuts and burns and thereby cleanly slices off the lesion or tumor pulled into said cylindrical lumen. Advantageously, the heat also seals any veins that may be present, preventing bleeding. The inner tube upon which RF blade 92 is mounted is electrically insulated from hollow needle 42.

As indicated in FIG. 9C, severed slice 79a is collected inside collecting bag or container 94 when the distal-to-proximal stroke of RF blade 92 has been completed. RF blade 92 and container 94 is then removed from the cylindrical lumen of hollow needle 42 and the severed specimen is taken to a lab for analysis.

As in the first three embodiments, a vacuum source may be used to pull tissue through slot 46 into the cylindrical lumen of hollow needle 42, or a balloon may be used to push the slot toward the tissue so that the tissue protrudes into said cylindrical lumen, or both a vacuum source and a balloon may be used in conjunction with one another.

The fifth embodiment, depicted in FIG. 10, has essentially the same structure as that of the fourth embodiment so most of the parts are marked by the same reference numerals. However, annular blade 92a is an ultrasonic cutting blade and ultrasonic connection 96a connects said ultrasonic cutting blade to ultrasound source 98a. Thus, the energy to accomplish the cutting by annular blade 92a is provided by ultrasound instead of RF energy, but in all other respects this fifth embodiment operates in the same way as the fourth embodiment.

Additional embodiments, although not depicted, are also within the scope of this invention. In lieu of actuating annular blade 92 with RF or ultrasound energy, other energy sources may be used as well. Light energy, such as laser, infrared, and the like, may also be used, for example.

In all embodiments, the cutting element may be extended and retracted by a vacuum, a motor means and gear train, a bias means, or any other suitable retracting and extending means. The cutting element may also be rotated by a suitable means as well, or controlled by any combination of means for retracting, extending, and rotating.

It will thus be seen that the objects set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

Now that the invention has been described,

1. A biopsy tool for removing large quantities of tissue in a brief period of time, comprising:
a hollow needle having a cylindrical lumen and a pointed distal end;
a slot of elongate, longitudinally-extending configuration formed in a cylindrical side wall of said hollow needle;
said slot having a distal end disposed proximal to said pointed distal end of said hollow needle;
an inner tube of hollow cylindrical configuration disposed within said lumen of said hollow needle;
rotation means for rotating said inner tube about its longitudinal axis;
an opening formed in said inner tube;
said opening being in intermittent registration with said slot as said inner tube rotates about its longitudinal axis;
a vacuum source in fluid communication with said lumen of said hollow needle so that tissue is pulled into said slot by said vacuum;
said opening having sharp peripheral edges so that tissue pulled into said slot by said vacuum is sheared off by a sharp peripheral edge of said opening as said opening rotates past said slot;
whereby a quantity of tissue is sliced off by said sharp peripheral edge during each rotation of said inner tube;
whereby each piece of sliced off tissue is pulled toward said vacuum source so that said slot and opening are clear of tissue for each rotation of said inner tube;
whereby a large quantity of tissue is removed by said biopsy tool in a brief amount of time;
whereby said biopsy tool eliminates a need to perform a biopsy and a tissue removal procedure in two separate steps; and
whereby each slice of tissue removed is in substantially undamaged condition so that it is suitable for use in a laboratory as a biopsy sample.

2. The biopsy tool of claim 1, wherein said opening is helical in configuration.

3. The biopsy tool of claim 1, wherein said opening is in the form of a straight slot having a longitudinal axis of symmetry disposed at an oblique angle to a longitudinal axis of said inner tube.

4. The biopsy tool of claim 1, wherein said opening has a diamond configuration.

5. The biopsy tool of claim 1, wherein said opening has a corkscrew configuration.

6. The biopsy tool of claim 1, wherein said opening has a sinusoidal configuration.

7. The biopsy tool of claim 1, wherein said opening has a sawtooth configuration.

8. The biopsy tool of claim 1, wherein said rotation means is a motor means, said motor means adapted to engage a proximal end of said inner tube.

9. The biopsy tool of claim 1, further comprising irrigating means for irrigating the tissue as it is sheared off.

10. The biopsy tool of claim 1, wherein said irrigating means includes a flow tube having a distal free end positioned near said slot formed in said hollow needle and a remote reservoir of liquid fluid that is in fluid communication with said flow tube.

11. The biopsy tool of claim 10, wherein said flow tube is mounted on an exterior surface of said hollow needle.

12. The biopsy tool of claim 10, wherein said flow tube is mounted within the lumen of said hollow needle.

13. The biopsy tool of claim 10, wherein said flow tube is formed within said cylindrical side wall of said hollow needle.

14. The biopsy tool of claim 1, further comprising:
a receptacle positioned between said vacuum source and said lumen of said hollow needle;
a filter trap disposed within said receptacle so that sheared tissue is drawn toward said vacuum source and captured in said filter trap.

15. The biopsy tool of claim 14, further comprising a housing for housing said motor means, said housing adapted to be held in a user's hand.

16. A biopsy tool for removing large quantities of tissue in a brief period of time, comprising:
a hollow needle having a cylindrical lumen and a pointed distal end;
a slot of elongate, longitudinally-extending configuration formed in a cylindrical side wall of said hollow needle;
said slot having a distal end disposed proximal to said pointed distal end of said hollow needle;
an inner tube of hollow cylindrical configuration disposed within said lumen of said hollow needle;
a motor means for rotating said inner tube about its longitudinal axis;
a hand-held housing for said motor means;
an opening formed in said inner tube;
said opening being in intermittent registration with said slot as said inner tube rotates about its longitudinal axis;
a vacuum source in fluid communication with said lumen of said hollow needle so that tissue is pulled into said slot by a vacuum;
said opening having sharp peripheral edges so that tissue pulled into said slot by said vacuum is sheared off by a sharp peripheral edge of said opening as said opening rotates past said slot;
a receptacle disposed between said lumen of said hollow needle and said vacuum source;
a filter trap disposed within said receptacle so that sheared tissue pulled from said lumen by said vacuum is captured by said filter trap;
whereby a quantity of tissue is sliced off by said sharp peripheral edge during each rotation of said inner tube;
whereby each piece of sliced off tissue is pulled toward said vacuum source so that said slot and opening are clear of tissue for each rotation of said inner tube;
whereby a large quantity of tissue is removed by said biopsy tool in a brief amount of time;
whereby said biopsy tool eliminates a need to perform a biopsy and a tissue removal procedure in two separate steps; and
whereby each slice of tissue removed is in substantially undamaged condition so that it is suitable for use in a laboratory as a biopsy sample.

17. The biopsy tool of claim 16, further comprising irrigating means for irrigating the tissue as it is sheared off.

18. The biopsy tool of claim 17, wherein said irrigating means includes a flow tube having a distal free end positioned near said slot formed in said hollow needle and a remote reservoir of liquid fluid that is in fluid communication with said flow tube.

19. The biopsy tool of claim 18, wherein said flow tube is mounted on an exterior surface of said hollow needle.

20. The biopsy tool of claim 18, wherein said flow tube is mounted within the lumen of said hollow needle.

21. The biopsy tool of claim 18, wherein said flow tube is formed within said cylindrical side wall of said hollow needle.

22. The biopsy tool of claim 18, wherein said housing for said motor housing includes a first and a second port, said first port providing fluid communication between said lumen of said hollow needle and said liquid fluid reservoir and said second port providing fluid communication between said lumen and said receptacle.

23. The biopsy tool of claim 16, wherein said filter trap is removable mounted within said receptacle so that said filter trap may be transported to a laboratory after said filter trap has collected a predetermined quantity of tissue.

24. A biopsy tool for removing large quantities of tissue from a lesion or tumor in a brief period of time, comprising:
a hollow needle having a cylindrical lumen and a pointed distal end;
a slot of elongate, longitudinally-extending configuration formed in a cylindrical side wall of said hollow needle;
said slot having a distal end disposed proximal to said pointed distal end of said hollow needle;
a balloon mounted in deflated configuration on an external surface of said hollow needle in diametrically opposed relation to said slot;
inflation means for inflating said balloon;
said balloon adapted to press against tissue when inflated, thereby urging said slot to press against a lesion or tumor so that said lesion or tumor enters into said slot and therefore into the cylindrical lumen of said needle;
an inner tube of hollow cylindrical configuration disposed within said cylindrical lumen of said hollow needle;
rotation means for rotating said inner tube about its longitudinal axis;
an opening formed in said inner tube, said opening having sharp peripheral edges so that a lesion or tumor pushed into said slot by said inflated balloon is sheared off by a sharp peripheral edge of said opening as said opening rotates past said slot;
said opening being in intermittent registration with said slot as said inner tube rotates about its longitudinal axis;
whereby inflation of said balloon urges said slot to press against said lesion or tumor to cause said lesion or tumor to enter into said cylindrical lumen of said hollow needle;
whereby a quantity of said lesion or tumor is sliced off by said sharp peripheral edge during each rotation of said inner tube;
whereby a large quantity of said lesion or tumor is removed by said biopsy tool in a brief amount of time;
whereby said biopsy tool eliminates a need to perform a biopsy and a lesion or tumor removal procedure in two separate steps; and
whereby each slice of lesion or tumor removed is in substantially undamaged condition so that it is suitable for use in a laboratory as a biopsy sample.

25. The biopsy tool of claim 24, further comprising:
a vacuum source in fluid communication with said cylindrical lumen of said hollow needle so that said lesion or tumor is pulled into said slot by said vacuum in conjunction with the pushing of said lesion or tumor into said slot by said inflated balloon;
whereby each piece of sliced off tissue is pulled toward said vacuum source so that said slot and opening are clear of tissue for each rotation of said inner tube.

26. A biopsy tool for removing large quantities of tissue in a brief period of time, comprising:
a hollow needle having a cylindrical lumen and a pointed distal end;
a slot of elongate, longitudinally-extending configuration formed in a cylindrical side wall of said hollow needle;
said slot having a distal end disposed proximal to said pointed distal end of said hollow needle;
a cutting cannula having an inner diameter sufficient to slidingly receive said hollow needle therein;
said cutting cannula having a sharp leading end;
motor means for reciprocating said cutting cannula along its longitudinal axis between a first retracted position where said slot is uncovered and a second extended position where said slot is covered by said cutting cannula;
a vacuum source in fluid communication with said lumen of said hollow needle so that a lesion or tumor is pulled into said slot by said vacuum;
said motor means moving said cutting cannula from its retracted position to its extended position only when said vacuum has pulled said lesion or tumor through said slot into said cylindrical lumen of said hollow tube;
said motor means maintaining said cutting cannula in said extended position until a severed piece of lesion or tumor is removed from said cylindrical lumen of said hollow needle by said vacuum;
whereby a quantity of tissue is sliced off by said sharp leading end during each reciprocation of said cutting cannula;
whereby each piece of sliced off tissue is pulled toward said vacuum source so that said slot is clear of tissue for each reciprocation of said cutting cannula;
whereby a large quantity of tissue is removed by said biopsy tool in a brief amount of time;
whereby said biopsy tool eliminates a need to perform a biopsy and a tissue removal procedure in two separate steps; and
whereby each slice of tissue removed is in substantially undamaged condition so that it is suitable for use in a laboratory as a biopsy sample.

27. A biopsy tool, comprising:
a hollow needle having a cylindrical lumen and a pointed distal end;
a slot of elongate, longitudinally-extending configuration formed in a cylindrical side wall of said hollow needle;
said slot having a distal end disposed proximal to said pointed distal end of said hollow needle;
a vacuum source in fluid communication with said cylindrical lumen of said hollow needle so that tissue is pulled through said slot into said cylindrical lumen by said vacuum;
an annular blade slidingly disposed within said cylindrical lumen of said hollow needle;
said annular blade having a retracted position where said annular blade is positioned distal of said slot and said annular blade having an extended position where said annular blade is positioned proximal of said slot;
a collecting bag connected to said annular blade on a distal side thereof for conjoint movement therewith;
said collecting blade having a closed bottom and an open mouth, said annular blade being positioned at said open mouth;
displacement means for displacing said annular blade and collecting bag from said retracted position to said extended position;
whereby a quantity of tissue is severed from a lesion or tumor by said annular blade when said annular blade is displaced from said retracted position to said extended position;
whereby said quantity of tissue severed from said lesion or tumor is collected by said collecting bag after it is fully severed;
whereby said collecting bag is removed from said cylindrical lumen of said hollow needle after said quantity of tissue has been severed; and
whereby the quantity of tissue severed from said lesion or tumor is in substantially undamaged condition so that it is suitable for use in a laboratory as a biopsy sample.

28. The biopsy tool of claim 27, wherein said annular blade is an RF blade and wherein said displacement means includes RF energy from an RF energy source connected to said annular blade.

29. The biopsy tool of claim 27, wherein said annular blade is an ultrasound blade and wherein said displacement means includes ultrasound energy from an ultrasound energy source connected to said annular blade.

30. The biopsy tool of claim 27, wherein said annular blade is a light-activated blade and wherein said displacement means includes light energy from a light energy source connected to said annular blade.

31. The biopsy tool of claim 30, wherein said light energy is laser energy.

32. The biopsy tool of claim 30, wherein said light energy is infrared energy.
